(19) United States
(12) Patent Application Publication
(10) Pub. No.: US 2003/0139355 A1
(43) Pub. Date: Jul. 24, 2003

(54) Medicine for orthopaedic surgical application and a treating method using thereof

(76) Inventors: Nobuaki Nakata, Kyoto-shi (JP); Tetsuo Yamamoto, Takatsuki-shi (JP); Atasushi Karino, Yamato-shi (JP); Kuniaki Kanatani, Kamo-gun (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/242,680

(22) Filed: Sep. 10, 2002

(30) Foreign Application Priority Data
Sep. 10, 2001 (JP) 2001-311755

(51) Int. Cl. A61K 31/704

(52) U.S. Cl. 514/33

(57) ABSTRACT

Disclosed are a medicine for treating an ischemia reperfusion disorder caused by a surgery in a field of orthopaedics, comprising glycyrrhizin and/or its pharmaceutically acceptable salts as an effective component and a treating method using the medicine.

### FIELD OF THE INVENTION

The present invention relates to a medicine for inhibiting ischemia reperfusion disorder resulting from surgery in the field of orthopaedics and to a treating method using the medicine.

### DESCRIPTION OF THE RELATED ART

Tissues of an organism are damaged by ischemia. In this case, it is known that reperfusion of a tissue after its ischemia will result in stronger damage of the tissue than that at the time of the ischemia. This phenomenon is called ischemia reperfusion disorder.

It is clinically known that ischemia reperfusion disorder takes place as a result of diseases specific to organs such as heart, liver, kidney and intestines or organ transplantation in the fields of internal medicine and surgery, or as a result of injury restoration surgery to blood vessel injury of a hand or foot in the field of orthopaedics. In particular, in orthopaedic operations, reperfusion subsequent to ischemia, such as that in the case where an air tourniquet has been used repeatedly for a long period of time, will cause a skeletal muscle disorder or edema of a subcutaneous tissue. If this continues for a significantly long period of time, there is a possibility that locally, arthrogryposis or reflex sympathetic dystrophy will be caused. However, no effective treating method for ischemia reperfusion disorder caused by surgeries in the field of orthopaedics has been established yet. In addition, under the present circumstances, it is tantamount to saying that there is no prophylactic for alleviating ischemia reperfusion disorder; development of an effective remedy at the earliest possible date has been awaited.

There has been a report on the sideration mechanism of ischemia reperfusion disorder. According to the report, at the time of ischemia reperfusion, activated platelets and adhesive molecules (selectins) expressed on vascular endothelial cells recognize sugar chains (Sialyl-Lewis X) on the surface of leukocytes to cause cell adhesion and subsequently thereto the permeability of micro blood vessels is enhanced so that neutrophiles, etc. can transmigrate to the outside of the blood vessel, with the result that tissue disorder accompanied by interstitial edema is caused [Am J. Physiol., 144(3), 592-598, 1994]. Actually, in the case where cell adhesion is inhibited by use of an antibody to a selectin, good improving effects on ischemia reperfusion disorder in an animal test model have been observed [SHOCK, 5(6), 402-407, 1996].

On the other hand, it has been reported that glycyrrhizin prevents binding between Sialyl-Lewis X and selectins in vitro [J. Biol. Chem., 269(31), 19663-19666, 1994], which suggests a possibility that it can be used as a medicine in anti-selectin treatment. Actually, there has also been reported that glycyrrhizin effectively exhibits inhibitory effects on ischemia reperfusion disorder in liver [Arch. Environ. Contam. Toxicol., 20, 432-436, 1991; Japan, J. Pharmacol., 58, 209-218, 1992].

Incidentally, glycyrrhizin and/or its pharmaceutically acceptable salts, either singly or as a blend with an amino acid such as glycine, cysteine, or methionine, have been provided for many years in medicine as an injection or perorally administered drug for improving liver function abnormality in chronic hepatitis or as a remedy for allergic diseases such as eczema or dermatitis, and has gained an established reputation for the safety.

However, thus far no study has been made as to whether or not glycyrrhizin and/or its pharmaceutically acceptable salts can be used as a remedy for ischemia reperfusion disorder caused by surgery in the field of orthopaedics, or there has been no report on the effect of glycyrrhizin and/or its pharmaceutically acceptable salts on inhibiting ischemia reperfusion disorder caused by surgery in the field of orthopaedics.

### SUMMARY OF THE INVENTION

An object of the present invention is to provide a medicine for use in inhibiting ischemia reperfusion disorder caused by surgery in a field of orthopaedics which is effective and high in safety while suppressing a side effect and a treating method using the medicine.

The inventors of the present invention have made extensive studies by using animal test models in order to achieve the aforementioned object and as a result they have found that glycyrrhizin and/or its pharmaceutically acceptable salts have action to inhibiting ischemia reperfusion disorder caused by surgery in the field of orthopaedics, thereby accomplishing the present invention.

That is, the present invention relates to:

(1) a medicine for inhibiting an ischemia reperfusion disorder caused by a surgery in a field of orthopaedics, including at least one of glycyrrhizin and its pharmaceutically acceptable salts as an effective component; and

(2) a medicine for inhibiting ischemia reperfusion disorder occurring in a muscle tissue or skin/subcutaneous tissue, including at least one of glycyrrhizin and its pharmaceutically acceptable salts as an effective component.

Also, according to the present invention, there is provided a method for treating an ischemia reperfusion disorder caused by a surgery in a field of orthopaedics, comprising administering a therapeutically effective amount of at least one of glycyrrhizin and its pharmaceutically acceptable salts to a patient for whom inhibition of the ischemia reperfusion disorder caused by the surgery in the field of orthopaedics is needed.

Also, according to the present invention, there is provided a method for treating an ischemia reperfusion disorder occurring in a muscle tissue or skin/subcutaneous tissue, comprising administering a therapeutically effective amount of at least one of glycyrrhizin and/or its pharmaceutically acceptable salts to a patient for whom inhibition of the ischemia reperfusion disorder occurring in the muscle tissue or skin/subcutaneous tissue is needed.

### DETAILED DESCRIPTION OF THE PREFERRED EMBODIMENTS

Hereinafter, the present invention will be illustrated in detail.

The medicine of the present invention that inhibits ischemia reperfusion disorder caused by surgery in the field of orthopaedics (hereinafter also referred to a “ischemia reperfusion disorder inhibito”) contains glycyrrhizin and/or its pharmaceutically acceptable salts as an active component.

Here, the contents of surgery in orthopaedics include operations under a tourniquet bandage that is repeatedly used for a long period of time and blood vessel injury restoration operations for a hand or foot. By the term “ischemia reperfusion disorder” caused by surgery in the field of orthopaedics is meant an ischemia reperfusion disorder occurring in a muscular tissue such as a hand or a foot, or skin or subcutaneous tissue and specifically includes skeletal muscle disorder, edema of skin or subcutaneous tissue, cell infiltration, or the like.

The glycyrrhizin of the present invention are compounds having a chemical structure represented by the following general formula (I)

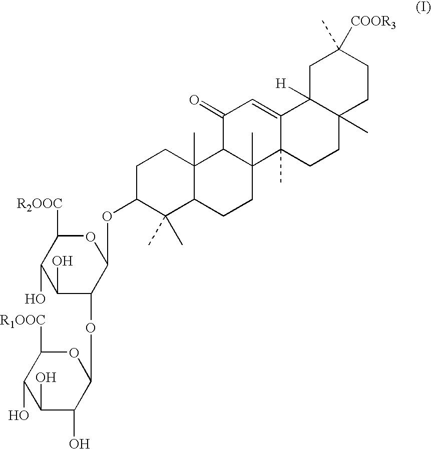

wherein R₁, R₂ and R₃ are each a hydrogen atom (H).

Its pharmaceutically acceptable salts are not particularly limited so far as they are physiologically acceptable as medicines. Preferred examples thereof include alkali metal salts such as sodium salts or potassium salts; alkaline earth metal salts such as calcium salts and magnesium salts; ammonium salts and organic amine salts such as triethylamine salts and triethanolamine salts; basic amino acids such as lysine salts and arginine salts; choline salts; etc. More specifically, compounds represented by the general formula (I) described above where R₁, R₂ and R₃ are all NH₄, Na, K, or the like, or where one or two of R₁, R₂ and R₃ are each H and the substituent(s) other than those is NH₄, Na, K, or the like.

The glycyrrhizin and/or its pharmaceutically acceptable salts of the present invention are used as ischemia reperfusion disorder inhibitors that contain them in amounts effective for exhibiting the effects of the present invention.

The preparation form of the ischemia reperfusion disorder inhibitor of the present invention is not particularly limited. It can be mixed with one kind or several kinds of harmless, generally pharmaceutically acceptable vehicles, carriers, excipients, binders, disintegrants, dispersants, lubricants, coatings, antiseptics, stabilizers, correctives, colorants or the like to form internal agents such as tablets, powder, granules, capsules, and liquors as well as injections such as sterile solutions and suspensions. These can be produced by use of conventionally known techniques.

For example, in the case where an injection is to be prepared, distilled water for injection or physiological saline is used as a solvent. Alternatively, this is mixed with dispersants, buffers, antiseptics, antioxidants, etc. as needed.

As the administration method, any one of oral and parenteral administrations may be selected.

The content of the glycyrrhizin and/or its pharmaceutically acceptable salts may be set optionally so far as ischemia reperfusion disorder inhibitory effects are generated. For example, the concentration of the glycyrrhizin and/or its pharmaceutically acceptable salts in an ischemia reperfusion disorder inhibitor is preferably from 0.1 mg/ml to 100 mg/ml, more preferably from 1 mg/ml to 50 mg/ml.

The time of intravenous administration of ischemia reperfusion disorder inhibitor of the present invention is preferably immediately before reperfusion and/or after from 30 minutes to 12 hours from reperfusion.

The ischemia reperfusion disorder inhibitor of the present invention can be effectively used not only for the treatment of but also for the prophylaxis of ischemia reperfusion disorder.

The present invention can provide a medicine having less possibility of causing side effects and being highly safe that is used for inhibiting ischemia reperfusion disorder caused by surgery in the field of orthopaedics.

### EXAMPLE

Hereinafter, the present invention will be illustrated in more detail by way of examples.

<Subject of Tests>

Seven rabbits (weighing 2.7 to 2.8 kg) were used as targets of tests.

<Medicine Used>

As the ischemia reperfusion disorder inhibitor containing glycyrrhizin and/or its pharmaceutically acceptable salts according to the present invention, an aqueous monoammonium glycyrrhizinate (containing 50 mg/ml of monoammonium glycyrrhizinate in the solution) and Glycyron Injection No.1 (manufactured by Minophargen Pharmaceutical Inc.) were used. The composition and properties of Glycyron Injection No.1 are as follows.

Composition: In 2 ml, the following components are contained.

(i) Monoammonium glycyrrhizinate 53 mg

(ii) Ammonia water prescribed in Japan Pharmacopoeia as an additive suitable amount

Properties: Colorless transparent aqueous injection (in ampoule)

(i) pH 6.0 to 7.4

(ii) Osmotic pressure about 1

<Test Procedure>

The above-mentioned rabbits were divided at random into a group administered with the ischemia reperfusion disorder inhibitor of the present invention (hereinafter also referred to as “medicine”) (n=3) and a group not administered with the medicine (n=4). These were further divided into phase A and phase B depending on a difference in the method of administration indicated below.

Phase A [ischemia 4.5 hours/reperfusion 48 hours: medicine-administered group (n=2) and medicine-non-administered group (n=3)

Phase B [ischemia 5.0 hours/reperfusion 24 hours: medicine-administered group (n=1) and medicine-non-administered group (n=1)].

The medicine-administered groups and -non-administered groups were named Examples 1 to 3 and Comparative Examples 1 to 4, respectively, as shown in Table 1.

TABLE 1

| | Experimental conditions | | | |
|---|---|---|---|---|
| | Medicine | | | |
| | | non- | | |
| | adminis- | administra- | ischemia | reperfusion |
| No. | tration | tion | (Time) | (Time) |

Phase A

| | | | | | |
|---|---|---|---|---|---|
| 1 | Example 1 | ◯ | | 4.5 | 48.0 |
| 2 | Example 2 | ◯ | | 4.5 | 48.0 |
| 3 | Comparative | | ◯ | 4.5 | 48.0 |
| | Example 1 | | | | |
| 4 | Comparative | | ◯ | 4.5 | 48.0 |
| | Example 2 | | | | |
| 5 | Comparative | | ◯ | 4.5 | 48.0 |
| | Example 3 | | | | |

Phase B

| | | | | | |
|---|---|---|---|---|---|
| 6 | Example 3 | ◯ | | 5.0 | 24.0 |
| 7 | Comparative | | ◯ | 5.0 | 24.0 |
| | Example 4 | | | | |

Ischemia was realized as follows. After anesthetizing the rabbits by intramuscular administration of celactal (R) and ketaral (R), avascularization was performed to the femoral region of the posterior limb by use of a tourniquet bandage. At a more proximal site, blood circulation was blocked by use of another tourniquet bandage.

In the medicine-administered group in phase A, 5 ml each of the aqueous monoammonium glycyrrhizinate was administered immediately before ischemia and immediately before reperfusion and further 12 ml of Glycyron Injection No.1 after 12 hours from the reperfusion in total three times through auricle vein and the progress was observed. The medicine-non-administered group in phase A was administered with the same amount of physiological saline and observed in the same manner.

In the medicine-administered group in phase B, 12 ml each of Glyceron Injection No.1 was administered immediately before ischemia, immediately before reperfusion and 30 minutes after reperfusion in total three times through auricle vein and the progress was observed. The medicine-non-administered group in phase B was administered with the same amount of physiological saline and observed in the same manner.

Evaluation of ischemia reperfusion disorder inhibitory action of the medicine of the present invention was performed by measuring the followings:

1) Change in peripheral diameter of foot articulation part and of femoral region under a tourniquet bandage proximal to the kneecap after 12 hours from the reperfusion,

2) Thickness of edema of skin/subcutaneous tissue of foot articulation part after 24 hours or 48 hours from the reperfusion, and

3) Degree of injury of tibialis anterior muscle after 24 hours or 48 hours from the reperfusion.

The rate of change of the peripheral diameter of foot articulation part and of femoral region under a tourniquet bandage proximal to the kneecap after 12 hours from the reperfusion was calculated by comparing peripheral diameters thereof before the ischemia and after 12 hours from the reperfusion.

The thickness of edema of skin/subcutaneous tissue of foot articulation part after 24 hours or 48 hours from the reperfusion was measured by taking an optical microscopic image (magnification ×1) of a hematoxylineosin (H&E) stained specimen obtained from a skin/subcutaneous tissue of foot articulation part, inputting the data in a computer, and measuring the maximum width thereon.

The degree of injury of tibialis anterior muscle after 24 hours or 48 hours from the reperfusion was obtained by classifying the region of muscle fiber that showed images of degeneration/necrosis of anterior tibialis and the amount of cell infiltration including neutrophiles in an H&E stained specimen into four grades based on the standards shown in Table 2 (grade 1, ±; grade 2, +; grade 3, ++; grade 4, +++) and evaluated by a third party pathologist doctor who did not participate in the tests.

TABLE 2

| | |
|---|---|
| Grade 1: ± | Degeneration was observed locally and in |
| | a very small number of muscle fibers and |
| | in that part a very small number of cell |
| | infiltrations were seen (the change was |
| | seen in 0-25% of the total range) |
| Grade 2: + | Degeneration was observed locally and in |
| | a small number of muscle fibers and in |
| | that part cell infiltrations were seen |
| | (the change was seen in 25-50% of the |
| | total range) |
| Grade 3: ++ | Degenerated muscle fibers increased and |
| | muscle fibers that lapsed into necrosis |
| | appeared now and then (the change was |
| | seen in 50-75% of the total range). |
| Grade 4: | Degeneration and necrosis of muscle |
| ++++ | fibers occurred in a wide range, and also |
| | cell infiltration increased (the change |
| | was seen in 75% or more of the total |
| | range) |

<Results>

Results are shown in Table 3.

TABLE 3

| | Experimental data | | | | | | | | | | | | |
|---|---|---|---|---|---|---|---|---|---|---|---|---|---|
| | | | | | | | | | Edema (mm) | | | | |
| | Peripheral diameter (cm) | | | | | | | | After reperfusion (48 hours) | | | | |
| | Before ischemia | | | | After reperfusion (12 hours) | | | | | | Non-isohemia | | |
| | Articulation part | | Femoral region | | Articulation part | | Femoral region | | Articulation part | | side | | |
| | Actual | | Actual | | Actual | Average | Actual | Average | Actual | Average | Actual | | |
| | Measure- | | Measure- | | Measure- | (Change | Measure- | (Change | Measure- | (Change | Measure- | Aver- | Evalu- |
| No. | ment | Average | ment | Average | ment | Rate) | ment | Rate) | ment | Rate) | ment | age | ation |

A

| | | | | | | | | | | | | | | |
|---|---|---|---|---|---|---|---|---|---|---|---|---|---|---|
| 1 | Ex- | 6.0 | 5.4 | 13.2 | 12.9 | 6.6 | 5.8 | 14.8 | 15 | 1.366 | 1.1513 | | 0.920 | + |
| | am- | | | | | | (7.4%) | | (16.3%) | | (64.5%) | | | |
| | ple 1 | | | | | | | | | | | | | |
| 2 | Ex- | 4.8 | | 12.6 | | 5.0 | | 15.2 | | 1.660 | | 0.920 | | + |
| | am- | | | | | | | | | | | | | |
| | ple 2 | | | | | | | | | | | | | |
| 3 | Com- | 5.5 | 5.2 | 13.9 | 12.9 | 6.8 | 5.9 | 18.0 | 17.1 | 3.523 | 4.089 | | 1.324 | ++ |
| | para- | | | | | | (13.5%) | | (32.6%) | | (208.8%) | | | |
| | tive | | | | | | | | | | | | | |
| | Ex- | | | | | | | | | | | | | |
| | am- | | | | | | | | | | | | | |
| | ple 1 | | | | | | | | | | | | | |
| 4 | Com- | 4.8 | | 12.1 | | 5.4 | | 15.5 | | 2.765 | | 1.308 | | + |
| | para- | | | | | | | | | | | | | |
| | tive | | | | | | | | | | | | | |
| | Ex- | | | | | | | | | | | | | |
| | am- | | | | | | | | | | | | | |
| | ple 2 | | | | | | | | | | | | | |
| 5 | Com- | 5.4 | | 12.7 | | 5.5 | | 17.8 | | 5.980 | | 1.340 | | ++ |
| | para- | | | | | | | | | | | | | |
| | tive | | | | | | | | | | | | | |
| | Ex- | | | | | | | | | | | | | |
| | am- | | | | | | | | | | | | | |
| | ple 3 | | | | | | | | | | | | | |

B

| | | Experimental data | | | | | | |
|---|---|---|---|---|---|---|---|---|
| | | Peripheral diameter (cm) | | | | | | |
| | | | | After reperfusion (12 hours) | | Edema (mm) | | |
| | | Before ischemia | | Articulation | | After reperfusion (48 hours) | | |
| | | Articulation | | part | Femoral region | Articulation | Non-ischemia | |
| | | part | Femoral region | Actual | Actual | part | side | |
| | | Actual | Actual | Measurement | Measurement | Actual | Actual | |
| No. | | Measurement | Measurement | (Change Rate) | (Change Rate) | Measurement | Measurement | Evaluation |
| 6 | Example 3 | 5.5 | 13.8 | 5.2 (−5.5%) | 14.8 (7.3%) | 1.021 | | ± |
| 7 | Comparative | 5.1 | 13.1 | 5.3 (3.9%) | 16 (22.1%) | 3.010 | 0.825 | +++ |
| | Example 4 | | | | | | | |

1) Observation with Unaided Eye

Immediately after reperfusion, the muscle in the anterior compartment on the outer surface of the leg swelled firm and this continued and became turgid in the medicine-non-administered group (Comparative Examples). After 12 hours, edemas around foot articulation part and Achilles tendon part were also seen and thereafter were in a tendency to gradually increase. Subcutaneous edema in the pressed part of femur and in foot articulation part was lighter in the medicine-administered group (Examples) than in the non-administered group (Comparative Examples) and also the hardness of the muscle in the anterior compartment of the outer surface of the leg seen immediately after reperfusion was in a gradually decreasing tendency.

2) The rate of change of the peripheral diameter of foot articulation part and of femoral region under a tourniquet bandage proximal to the kneecap after 12 hours from the reperfusion.

The peripheral diameter of foot articulation part before and after ischemia reperfusion in phase A was in average 5.4 cm before ischemia in the medicine-administered group while after 12 hours from the reperfusion it was 5.8 cm (Examples 1 and 2). In terms of the rate of change of the peripheral diameter, this was 7.4%. On the other hand, in the non-administered group, the peripheral diameter of the foot articulation part before and after the ischemia reperfusion was in average 5.2 cm before the ischemia in contrast to 5.9 cm after 12 hours from the reperfusion (Comparative Examples 1 to 3). The rate of change in peripheral diameter was 13.5%. The swelling of foot articulation part after 12 hours from the reperfusion was more inhibited in the medicine-administered group (Examples) than in the non-administered group (Comparative Examples).

The peripheral diameter of femoral region under a tourniquet bandage proximal to the kneecap before and after ischemia reperfusion in phase A was in average 12.9 cm before ischemia in the medicine-administered group while after 12 hours from the reperfusion it was 15.0 cm (Examples 1 and 2). In terms of the rate of change of the peripheral diameter, this was 16.3%. On the other hand, in the non-administered group, the peripheral diameter of the femoral region under a tourniquet bandage proximal to the kneecap before and after the reperfusion was in average 12.9 cm before the ischemia in contrast to 17.1 cm after 12 hours from the reperfusion (Comparative Examples 1 to 3). The rate of change in peripheral diameter was 32.6%. The swelling of the femoral region under a tourniquet bandage proximal to the kneecap after 12 hours from the reperfusion was more inhibited in the medicine-administered group (Examples) than in the non-administered group (Comparative Examples).

The peripheral diameter of foot articulation part before and after ischemia reperfusion in phase B was 5.5 cm before ischemia in the medicine-administered group while after 12 hours from the reperfusion it was 5.2 cm (Example 3). In terms of the rate of change of the peripheral diameter, this was ×5.5%. On the other hand, in the non-administered group, the peripheral diameter of the foot articulation part before and after the ischemia reperfusion was 5.1 cm before the ischemia in contrast to 5.3 cm after 12 hours from the reperfusion (Comparative Example 4). The rate of change in peripheral diameter was 3.9%. The swelling of foot articulation part after 12 hours from the reperfusion was more inhibited in the medicine-administered group (Examples) than in the non-administered group (Comparative Examples).

The peripheral diameter of the femoral region under a tourniquet bandage proximal to the kneecap before and after ischemia reperfusion in phase B was 13.8 cm before ischemia in the medicine-administered group while after 12 hours from the reperfusion it was 14.8 cm (Example 3). In terms of the rate of change of the peripheral diameter, this was 7.3%. On the other hand, in the non-administered group, the peripheral diameter of the femoral region under the a tourniquet bandage proximal to the kneecap before and after the ischemia reperfusion was 13.1 cm before the ischemia in contrast to 16.0 cm after 12 hours from the reperfusion (Comparative Example 4). The rate of change in peripheral diameter was 22.1%. The swelling of femoral region under a tourniquet bandage proximal to the kneecap after 12 hours from the reperfusion was more inhibited in the medicine-administered group (Examples) than in the non-administered group (Comparative Examples).

3) Thickness of edema of skin/subcutaneous tissue of foot articulation part after 24 hours or 48 hours from the reperfusion

The thickness of edema of skin/subcutaneous tissue of foot articulation part after 48 hours from the reperfusion in phase A was in average 1.513 mm in the medicine-administered group. In terms of rate of change taking the non-ischemia side as a control, this was 64.5% (Examples 1 and 2). On the other hand, in the non-administered group, the thickness of edema of skin/subcutaneous tissue of foot articulation part after 48 hours from the reperfusion was in average 4.089 mm and the rate of change taking the non-ischemia side as a control was 208.8% (Comparative Examples 1 to 3). The thickness of edema in the skin/subcutaneous tissue of foot articulation part after 48 hours from the reperfusion was more inhibited in the medicine-administered group (Examples) than in the non-administered group (Comparative Examples).

The thickness of edema of skin/subcutaneous tissue of foot articulation part after 24 hours from the reperfusion in phase B was 1.021 mm in the medicine-administered group (Example 3). On the other hand, in the non-administered group, the thickness of edema of skin/subcutaneous tissue of foot articulation part after 24 hours from the reperfusion was 3.010 mm (Comparative Example 4). The thickness of edema in the skin/subcutaneous tissue of foot articulation part after 24 hours from the reperfusion was more inhibited in the medicine-administered group (Examples) than in the non-administered group (Comparative Examples).

4) Degree of injury of tibialis anterior muscle tissue after 24 hours or 48 hours from the reperfusion

The region of muscle fiber that showed degeneration/necrosis of tibialis anterior muscle and the amount of cell infiltration including neutrophiles, etc. into the muscle tissue after ischemia reperfusion were observed. As a result, in the H&E stained specimens, only Rank 1 (±) and Rank 2 (+) were observed in the medicine-administered group (Examples) while in the medicine-non-administered group, Ranks 2 (+) to 4 (+++) were observed. As compared with the non-administered group (Comparative Examples), the medicine-administered group (Examples) apparently showed a reduction of the region of degeneration/necrosis of muscle fiber after ischemia reperfusion and an inhibited cell infiltration into the muscle tissue. Further, with an increasing degree of ischemia reperfusion disorder, the region of muscle fiber that showed vacuolar degeneration or necrosis expanded and there were intensively observed expansion of intervals between muscle fibers and between muscle fiber bundles, hyperplasia of collagen fibers, and cell infiltration which are observations indicating edema. In addition, expansion of the range of degeneration/necrosis of muscle fibers and the infiltration amount of cells including neutrophiles coincided well. In particular, in phase B (ischemia 5.0 hours/reperfusion 24 hours), clear inhibitory effects on the region of degeneration/necrosis of tibialis anterior muscle and the infiltration amount of cells such as neutrophiles into the muscle tissue after ischemia reperfusion were observed in the medicine-administered group (Example 3).

As will be apparent from the results of experiments, an ischemia reperfusion disorder inhibiting action is observed in the glycyrrhizin and/or its pharmaceutically acceptable salts and hence medicines containing such as an effective component can be effectively used as ischemia reperfusion disorder inhibitors.

What is claimed is:

1. A method for treating an ischemia reperfusion disorder caused by a surgery in a field of orthopaedics, comprising administering a therapeutically effective amount of at least one of glycyrrhizin and its pharmaceutically acceptable salts to a patient for whom inhibition of the ischemia reperfusion disorder caused by the surgery in the field of orthopaedics is needed.

2. A method for treating an ischemia reperfusion disorder occurring in a muscle tissue or skin/subcutaneous tissue, comprising administering a therapeutically effective amount of at least one of glycyrrhizin and its pharmaceutically acceptable salts to a patient for whom inhibition of the ischemia reperfusion disorder occurring in the muscle tissue or skin/subcutaneous tissue is needed.
